(19) United States
(12) Patent Application Publication
(10) Pub. No.: US 2010/0210717 A1
(43) Pub. Date: Aug. 19, 2010

(54) PHARMACEUTICAL COMPOSITIONS COMPRISING MYCOPHENOLIC ACID OR MYCOPHENOLATE SALT

(76) Inventors: Jürgen Dederichs, Neuenburg (DE); Thomas Rigassi, Therwil (CH)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/772,608

(22) Filed: May 3, 2010

Related U.S. Application Data
(63) Continuation of application No. 10/492,289, filed on Sep. 2, 2004, now abandoned.

(30) Foreign Application Priority Data
Oct. 17, 2001 (GB) 0124953.1

(51) Int. Cl. A61K 31/365; A61K 9/28; A61P 37/06

(52) U.S. Cl. 514/470; 424/474

(57) ABSTRACT

The invention provides a solid dosage form, e.g. a tablet, comprising mycophenolic acid or mycophenolate salt and a process for its production.

The present invention relates to novel pharmaceutical compositions comprising mycophenolic acid or mycophenolate salt.

Mycophenolic acid, also referred to herein as MPA, a natural product of complex structure and particular sensitivity, was first isolated in 1896 and has been disclosed to have anti-tumor, anti-viral, immunosuppressive, anti-psoriatic, anti-inflammatory, and anti-cancer activity over 15 years ago. Attempts have been made to increase the bioavailability of MPA by making high molecular weight derivatives such as the morpholinomethylester of MPA, also known as mycophenolate mofetil which is being commercially used as an immunosuppressant for the treatment or prevention of organ or tissue transplant rejection. WO97/38689 and U.S. Pat. No. 6,025,391 describe a pharmaceutical composition comprising a mycophenolate salt, the composition being adapted to release the mycophenolate salt in the upper part of the intestinal tract. Capsules as representative unit dosage forms are disclosed. We have now found that these compositions are effective and tolerated for immunosuppressive indications in clinical trials.

Despite MPA being known since 1896, however, there still exists a need for commercially acceptable mycophenolic acid or mycophenolate salt dosage forms for oral administration with good patient convenience and acceptance.

In accordance with the present invention it has now surprisingly been found that particularly suitable pharmaceutical compositions comprising mycophenolic acid or mycophenolate salt having particularly interesting bioavailability characteristics, being well-tolerated, stable, and convenient to administer, are obtainable when the compositions are formulated as oral solid dosage forms, preferably in the form of tablets.

Difficulties in the formulation of oral solid dosage forms, e.g. tablets, comprising mycophenolic acid or mycophenolate salt, can be for example the low bulk density of the drug substance, resulting in e.g. low mechanical stability of the solid dosage form and/or unfavourable size particularly when a high amount of excipients or additives is added to improve the mechanical stability properties. Tablets with inferior mechanical properties are liable to crumble, edge chipping or break. These difficulties are even greater when it is desirable to use an oral solid dosage form with a high drug loading. Moreover, for certain groups of patients, oral administration of large tablets is either undesirable or impractical.

It has now been found that pharmaceutically acceptable oral solid dosage forms, e.g. in the form of tablets, of mycophenolic acid or mycophenolate salt are obtainable in a favourable small and mechanically stable form with a high drug loading. Oral dosage forms being particularly convenient to administer and stable, may be obtained, e.g. by preparation of tablets by compression methods. More specifically, the tablets of the invention may be prepared by granulation followed by compression methods.

Whereas hereinafter the compositions of the invention are described with particular reference to tablets, other types of oral solid dosage forms, e.g. effervescent tablets, fast dispersible tablets, matrix tablets, minitablets, multilayer tablets, pulsed release tablets, pellets, capsules, granulates or powder form, e.g. in a sachet or a bottle, may be produced and are encompassed within the scope of this invention.

Accordingly, in one aspect the present invention provides a solid dosage form comprising a pharmacologically effective amount of mycophenolic acid or mycophenolate salt,

wherein the mycophenolic acid or mycophenolate salt is present in an amount of from about 20% to about 95%, e.g. at least about 35, 40, 45, 50 or 55% to about e.g 60, 65, 70, 75, 80% or e.g 35 to 55% by weight, preferably more than 55% by weight based on the total weight of the solid dosage form (total solid dosage form weight being e.g the core with any coating). In particular the amount of mycophenolic acid or mycophenolate salt may be from 45 to 80% by weight, e.g. 50 to 65% by weight based on the total weight of the solid dosage form.

More particularly, the present invention provides a tablet comprising
(a) a pharmacologically effective amount of mycophenolic acid or mycophenolate salt, and
(b) pharmaceutically acceptable additives suitable for the preparation of tablets by compression methods
   wherein the mycophenolic acid or mycophenolate salt is present in an amount of from about 20% to about 90%, e.g. at least about 35, 40, 45, 50 or 55% to about e.g 60, 65, 70, 75, 80% or e.g 35 to 55% by weight, preferably more than 55% by weight based on the total weight of the tablet (total tablet weight being e.g the core with any coating). In particular the amount of mycophenolic acid or mycophenolate salt may be from 45 to 80% by weight, e.g. 50 to 65% by weight based on the total weight of the tablet.

The term “pharmacologically effective amount”, as used herein, is understood to mean the amount of active agent which halts or reduces the progress of the condition to be treated or which otherwise completely or partly cures or acts palliatively on the condition. Such an amount can be routinely determined by routine experimentation.

The tablets of the invention are, despite the high drug loading of at least about 20% by weight based on the total weight of the tablet, small, and, therefore, convenient to administer. Furthermore, the tablets of the invention were found to be stable, e.g. during storage, handling or packaging, effective and well-tolerated. Furthermore, the tablets have improved mechanical properties; e.g. when uncoated tablets are scored, they are easy to divide to produce e.g. half doses.

In addition, the tablets obtained are stable both to the production process and during storage, e.g. for 2 years or even 3 years in conventional packaging, e.g. sealed aluminium blister packs. Less than about 5%, e.g. 2 or less of mycophenolic acid or mycophenolate salt may degrade during this time as determined in conventional, e.g. stress, tests.

In another embodiment this invention provides a tablet comprising from 50 mg to 500 mg mycophenolic acid or mycophenolate salt, e.g. of from 100 mg to about 500 mg mycophenolic acid or mycophenolate salt, preferably from about 180 to about 360 mg mycophenolic acid or from about 190 to about 385 mg mycophenolate salt.

When mycophenolate salt is used, cationic salts of MPA, e.g. alkali metal salts, especially the sodium salt, alkaline earth metal salts, an ammonium salt or a salt with an organic base may be used. According to the present invention, preferably the mono-sodium salt may be used. This may be obtained in crystalline form by recrystallization, e.g. from acetone/ethanol if necessary with water; m.p. 189-191° C.

In another aspect, the present invention provides an oral solid dosage form, e.g. tablet, wherein the mycophenolate sodium salt is in crystalline form.

In accordance with the present invention it has surprisingly been found that it may be especially advantageous to use the MPA or mycophenolate salt in the form of its anhydrate. Preferably the tablet according to the invention contains less than 5%, more preferably less than 2%, e.g. down to 0.1 or 0.3% MPA or mycophenolate salt in form of its hydrate.

Accordingly, in a further aspect the present invention provides a tablet comprising a pharmacologically effective amount of mycophenolic acid or mycophenolate salt in substantially anhydrous form. The term “in substantially anhydrous form”, as used herein, is understood to mean “in an amount of about 95%, preferably about 98% anhydrous form”.

In another aspect the present invention provides a tablet containing from about 50 mg to about 500 mg, preferably 100 mg to 500 mg, of crystalline mycophenolate mono sodium salt in anhydrous form.

Following pharmaceutically acceptable additives may be present in the tablets, e.g.

(1.1) one or more fillers, e.g. lactose, e.g. anhydrous lactose;
(1.2) one or more disintegrants, e.g. maize starch, Crospovidone®, or carboxymethylcellulose (CMC)-Ca;
(1.3) one or more binders, e.g. polyvinypyrrolidone, e.g. commercially as Povidone® K30;
(1.4) one or more glidants, e.g. colloidal silicon dioxide, e.g. commercially available as Aerosil® 200;
(1.5) one or more lubricants, e.g. magnesium stearate.

Reference is made to the extensive literature on the subject for these and other excipients and procedures mentioned herein, see in particular Handbook of Pharmaceutical Excipients, Second Edition, edited by Ainley Wade and Paul J. Weller, American Pharmaceutical Association, Washington, USA and Pharmaceutical Press, London; and Lexikon der Hilfsstoffe für Pharmazie, Kosmetik and angrenzende Gebiete edited by H. P. Fiedler, 4th Edition, Editio Cantor, Aulendorf and earlier editions which are incorporated herein by reference.

As fillers or diluents (1.1) we contemplate in particular sugar e.g. confectioner's sugar, compressible sugar, dextrates, dextrin, dextrose, lactose, e.g. lactose anhydrous, mannitol, microcrystalline cellulose, in particular having a density of about 0.45/cm³, e.g. commercially available as Avicel®, for example from FMC Corp., powdered cellulose, starches, e.g. maize starch, calcium phosphates, e.g. dibasic calcium phosphate dihydrate, sorbitol, sucrose and talc. Preferably lactose anhydrous may be used.

As disintegrants (1.2) maize starch, e.g. pre-gelatinized maize starch; sodium starch glycolate; croscarmellose sodium; CMC-Ca; CMC-NA; crosslinked PVP, e.g. commercially available as Crospovidone®, Polyplasdone®, from the ISP company, or Kollidon® XL; alginic acid; sodium alginate; or guar gum may be used. Preferably maize starch; crosslinked PVP, e.g. Crospovidone®; crosslinked CMC; or croscarmellose sodium, e.g. commercially available as Ac-di-sol® from FMC Corp., may be used. In particular a mixture of maize starch and crosslinked PVP may be used, e.g. in a weight ratio of from 1:1 to 1:5.

As binders (1.3) are contemplated particularly starches, e.g. potato starch, wheat starch, corn starch; microcrystalline cellulose, e.g. products known as Avicel®, Filtrak®, Heweten® or Pharmacel®; hydroxypropyl cellulose; hydroxyethyl cellulose; hydroxypropylmethyl cellulose; e.g. hydroxypropyl cellulose having a hydroxypropyl content of 5 to 16% by weight and a Mw of from 80 000 to 1 150 000, more particularly 140 000 to 850 000; or polyvinylpyrrolidone, e.g. commercially available as Povidone® K30 from BASF. Preferably Povidone® K30 may be used.

Examples of glidants (1.4) include e.g. colloidal silica, e.g. colloidal anhydrous silica, e.g. Aerosil® 200, magnesium trisilicate, powdered cellulose, starch, talc or tribasic calcium phosphate. Preferably Aerosil® 200 may be used.

Examples of lubricants (1.5) include e.g. Mg, Al or Ca stearate, PEG 4000-8000, hydrogenated castor oil, glyceryl monostearate or talc. Preferably magnesium stearate may be used.

One or more of these additives can be selected and used having regard to the particular desired properties of the tablet by routine experimentation.

The amount of each type of additive employed, e.g. filler or diluent, disintegrant, binder glidant or lubricant, may be readily ascertained using procedures conventional in the art. Thus for example, the amount of filler or diluent (1.1) may vary within a range of from 5 to 40% by weight, e.g. 10 to 20% by weight;

the amount of disintegrant (1.2) may vary within a range of from 2 to 20% by weight, e.g. 10 to 15% by weight;
the amount of binder (1.3) may vary within a range of from about 1 to 45% by weight, e.g. 2 to 30% by weight, in particular 5 to 10% by weight;
the amount of glidant (1.4) may vary within ranges of from 0.1 to 10% by weight, in particular 0.1 to 5% by weight, e.g. 0.5 to 3% or 2 to 4% by weight;
the amount of lubricant (1.5) may vary within a range of from 0.1 to 5.0% by weight, e.g. 0.5 to 2% by weight;
based on the total weight of the tablet.

It will be appreciated that any given excipient may serve more than one function e.g. as filler or diluent, disintegrant, binder glidant, and/or lubricant. The upper range of binder is preferably used in case of matrix tablets.

Preferably the oral dosage form of the invention, e.g. the tablet, contains as active ingredient only MPA or mycophenolate salt.

It is a characteristic of the tablet according to the invention that despite the high content of MPA or mycophenolate salt, it contains only a relatively small amount of additives. This advantageously results in a mechanically stable tablet having a small size. The total amount of additives in a given unit dosage may be about 65% or less by weight based on the total weight of the tablet, more particularly about 50% or less. Preferably the additive content is in the range of about 35 to 55% by weight, more particularly 45 to 55% by weight, e.g. 38 to 43% by weight.

The absolute amounts of each additive and the amounts relative to other additives is similarly dependent on the desired properties of the tablet and may also be chosen by routine experimentation. For example, the tablet may be chosen to exhibit accelerated and/or delayed release of the MPA or mycophenolate salt with or without quantitative control of the release of active agent. Preferably the tablet is chosen to exhibit delayed release of the mycophenolate salt, e.g. the mycophenolate mono-sodium salt.

Thus, where accelerated release is desired, e.g. about 90% release within 10 minutes, more particularly five minutes, a disintegrant such as crosslinked polyvinyl pyrrolidone, for example those products known as Polyplasdone®XL or Kollidon®CL, in particular having a molecular weight in excess of 1 000 000, more particularly having a particle size distribution of less than 400 microns or less than 74 microns, or reactive additives (effervescent mixtures) that effect rapid disintegration of the tablet in the presence of water, for example so-called effervescent tablets that contain an acid in solid form, typically citric acid, which acts in water on a base containing chemically combined carbon dioxide, for example sodium hydrogen carbonate or sodium carbonate, and releases carbon dioxide, may be used.

Whereas if delayed release is desired one can employ pellet coating technology, wax matrix systems, polymer matrix tablets or polymer coatings conventional in the art. Preferably a coating technology may be used.

Quantitative control of the release of the MPA or mycophenolate salt can be achieved by conventional techniques known in the art. Such dosage forms are known as oral osmotic systems (OROS), coated tablets, matrix tablets, press-coated tablets, multilayer tablets and the like. According to the present invention preferably coated tablets may be used.

In a tablet according to the present invention, preferred additives are Mg stearate, anhydrous colloidal silica, maize starch, polyvinylpyrrolidone, crosslinked polyvinylpyrrolidone and anhydrous lactose. The amounts of additive employed will depend upon how much MPA or mycophenolate salt is to be used. The stearate, e.g. Mg stearate is preferably employed in amounts of 0.1 to 5.0% by weight, e.g. 0.5% to 2.0% by weight. The silica is preferably employed in an amount of from 0.1 to 10% by weight, e.g. 0.5% to 5.0% by weight.

In a further aspect the invention provides a tablet as described above wherein the tablet is enteric coated. The enteric coating may be applied not only to tablets but also to other oral dosage forms such as indicated above, e.g. granules, which may be further compressed to tablets, or to the MPA or mycophenolate salt drug substance.

The term “enteric coating”, as used herein, comprises any pharmaceutically acceptable coating preventing the release of the active agent in the stomach and sufficiently disintegrating in the intestinal tract, preferably in the upper part of the intestinal tract (by contact with approximately neutral or alkaline intestine juices) to allow the resorption of the active agent through the walls of the intestinal tract. In vitro tests for determining whether or not a coating is classified as an enteric coating have been published in the pharmacopoeia of various countries.

More specifically, the term “enteric coating”, as used herein, refers to a coating which remains intact for at least 2 hours, in contact with artificial gastric juices such as HCl of pH-1 at 36 to 38° C. and preferably thereafter disintegrates within 30 minutes in artificial Intestinal juices such as a KH₂PO₄ buffered solution of pH 6.8.

The thickness of the coating may vary and depends inter alia on its permeability in water and acids. A typical coating may be about 20 to 80 mg, e.g. 30 to 70 mg, e.g. about 65 mg on a 685 mg uncoloured tablet.

In general satisfactory results are obtained with a coating of 50-200 microns, preferably 75-150 microns thickness. The coating is suitably selected from macromolecular polymers. Suitable polymers are listed in e.g. L. Lachman et al. The Theory and Practice of Industrial Pharmacy, 3rd Ed, 1986, p. 365-373, H. Sucker et al, Pharmazeutische Technologie, Thieme, 1991, p. 355-359, Hagers Handbuch der pharmazeutischen Praxis, 4th Ed. Vol. 7, pages 739 to 742 and 766 to 778, (Springer Verlag, 1971) and Remington's Pharmaceutical Sciences, 13th Ed., pages 1689 to 1691 (Mack Publ., Co., 1970) and comprise e.g. cellulose ester derivatives, cellulose ethers, acrylic resins such as methylacrylate copolymers, copolymers of maleic acid and phthalic acid derivatives, shellac, hydroxypropylmethylcellulose acetate succinate, or polyvinylacetate phthalate.

The preferred films are made from cellulose acetate phthalate and trimellitate; methacrylic acid copolymers, e.g. copolymers derived from methacrylic acid and esters thereof, containing at least 40% methacrylic acid; and especially hydroxypropyl methylcellulose phthalate, e.g. as available under the name hypromellose phthalate or hydroxypropyl methylcellulose phthalate HP50 from e.g. Shin-Etsu.

Polymethacrylates include those of molecular weight above 100,000 daltons based on, e.g. methacrylic acid and methyl or ethyl methacrylate in a ratio of about 1:1. Typical products include Eudragit L, e.g. L 100-55, or Eudragit S marketed by Röhm GmbH, Darmstadt, Germany. A mixture adapted for release of the active agent in the upper part of the intestinal tract, e.g. Eudragit L and S, may also be used. Eudragit L is preferred.

Typical cellulose acetate phthalates have an acetyl content of 17-26% and a phthalate content of from 30-40% with a viscosity of ca. 45-90 cP. An example of an appropriate cellulose acetate phthalate is the marketed product CAP (Eastman Kodak, Rochester N.Y., USA).

Typical cellulose acetate trimellitates have an acetyl content of 17-26%, a trimellityl content from 25-35% with a viscosity of ca. 15-20 cS. An example of an appropriate cellulose acetate trimellitate is the marketed product CAT (Eastman Kodak Company, USA).

Hydroxypropylmethyl cellulose phthalates, typically have a molecular weight of from 20,000 to 100,000 daltons e.g. 80,000 to 130,000 daltons, e.g. a hydroxypropyl content of from 5 to 10%, a methoxy content of from 18 to 24% and a phthalyl content from 21 to 35%.

Examples of suitable hydroxypropyl methylcellulose phthalates (HPMCP) are the marketed products having a hydroxypropyl content of from 6-10%, a methoxy content of from 20-24%, a phthalyl content of from 21-27%, a molecular weight of about 84,000 daltons known under the trade mark HP50 and available from Shin-Etsu Chemical Co. Ltd., Tokyo, Japan, and having a hydroxypropyl content, a methoxy content, and a phthalyl content of 5-9%, 18-22% and 27-35% respectively, and a molecular weight of 78,000 daltons, known under the trademark HP55 and available from the same supplier.

A preferred coating is HPMCP HP50.

The enteric coating may be carried out in conventional manner, e.g. so that the cores are sprayed with a solution of the enteric-coating. The term “core”, as used herein, is understood to mean tablets, granules, pellets or MPA or mycophenolate salt drug substance. Attention is drawn to the numerous known methods of coating employed in the art, e.g. spray coating in a fluidized bed, e.g. by the known methods using apparatus available from Aeromatic, Glatt, Wurster or Hüttlin, in a perforated pan by the Accela Cota method, or to the submerged sword coating method. The additives commonly used in confectioning are employed in such methods.

Suitable solvents for the enteric-coating are for example organic solvents, e.g. an alcohol such as ethanol or a mixture of alcohols, e.g. ethanol and isopropanol, a ketone such as acetone, halogenated hydrocarbons such as CH₂Cl₂ or mixtures of such solvents, e.g. ethanol/acetone, e.g. 1:1 to 10:1, wherein the ethanol part may contain up to 5% isopropanol.

If desirable a softener such as di-n-butylphthalate or triacetin may be added to such a solution, e.g. in a ratio of coating material to softener of from 1:about 0.05 to about 0.3.

Enteric coating material, e.g. polymethacrylates such as disclosed above or other acidic coating materials may also be applied from an aqueous medium. If desired for cellulose phthalates and other acidic coating materials, a water soluble salt, e.g. an ammonium salt, may be formed and an aqueous solution thereof may be used.

According to the invention, it has been found that it may be advantageous to employ MPA or mycophenolate salt in its anhydrous form. Difficulties encountered in the coating of tablets comprising MPA or mycophenolate salt in anhydrous form using an aqueous solvent may include hydrate formation, e.g. various degrees of hydration, and the formation of polymorphs of the drug substance.

In order to meet these and related difficulties, it has now surprisingly been found that a non-aqueous coating, e.g. an organic coating, may be used.

According to a preferred embodiment, enteric coated tablets comprising MPA or mycophenolate salt in their anhydrous form may be obtained using an organic coating by optimization of the amount of coating material and/or optimization of the spraying conditions during the coating process. The absolute amount of the coating material and the spraying conditions may be selected and used having regard to the particular desired properties of the tablet by routine experimentation.

Accordingly, in another embodiment the present invention provides a process for the preparation of an enteric coated tablet comprising MPA or mycophenolate salt in anhydrous form, which process comprises dissolving/dispersing the coating material, e.g. a hydroxypropyl methylcellulose phthalates, e.g. HPMCP HP50, and optionally pigments, e.g. iron oxide, indigotine, e.g. indigotine lake, and/or titanium dioxide, in an organic solvent or a mixture of organic solvents, e.g. ethanol/acetone (w/w), and spraying the solution/dispersion onto the tablets.

In yet a further aspect the present invention provides a method of avoiding hydrate formation comprising dissolving/dispersing the organic coating material in an organic solvent or a mixture of organic solvents, e.g. as already disclosed above, and spraying the solution/dispersion onto the tablets.

Preferably the coating material may be used in an amount of from about 5 to 20% by weight, e.g. about 10 to 15% by weight, preferably about 10% by weight based on the total weight of the film-coated tablet.

Desirably, the organic solvent, e.g. ethanol, is substantially anhydrous, containing e.g. less than 15%, more preferably less than 10%, most preferably less than 5% water. Suitable as ethanol may be ethanol 94% (w/w) or absolute ethanol.

A fluidized bed costar or a perforated pan coater may be used for coating.

Conveniently the cores are treated at room temperature or warmed up to 40° C. e.g. by means of warm air of 40° up to 70° C., before spraying. To avoid a sticking of the cores the spray procedure is preferably interrupted at certain time intervals and the cores then warmed up again. It is, however, also possible to proceed without interruption of the spray procedure, e.g. by automatic regulation of the spray amount taking into account the temperature of exhaust air and/or cores.

The spray pressure may vary within wide ranges, in general satisfactory results are obtained with a spray pressure of from about 1 to about 70 bar, e.g. of from about 20 to about 60 bar, preferably of from about 40 to about 50 bar, e.g for an airless spray system.

The invention provides in another of its aspects a process of making tablets as hereinabove described. Such tablets may be produced by
(i) mixing the mycophenolic acid or mycophenolate salt and pharmaceutically acceptable additives,
(ii) subjecting a mixture obtained in step (i) to granulation
(iii) compressing the granulates obtained in step (ii) and pharmaceutically acceptable additives to form the tablet.

The granulation step (ii) may be a wet-granulation, e.g. spray granulation or high shear mixing methods, melt granulation or dry granulation, e.g. roller compaction.

The process as described above may further comprise coating of the mycophenolic acid or mycophenolate salt, and/or granulate, and/or tablet. The coating procedure may be performed according to a process as specified above.

In another aspect of the invention the present invention provides a granulate produced by a process as set out in steps (i) and (ii) as defined above.

When MPA and mycophenolate salt are used in their anhydrous form all steps as specified above, particularly the granulation step (ii) and the coating procedure may be carried out applying non-aqueous solvents only, e.g. organic solvents, e.g. as specified above. Preferably the organic solvent, e.g. ethanol, is substantially anhydrous, e.g. as specified above.

Tablets of the invention may also be produced by direct compression of drug substance and additives comprising step (i) and step (iii), in the absence of step (ii).

In case of unfavourable drug substance properties, e.g. a low bulk density of the drug substance, granulation techniques such as melt granulation, wet granulation or roller compaction may be performed, followed by the compression step. More specifically, in one aspect the present invention provides a process comprising

(i) mixing MPA or mycophenolate salt and pharmaceutically acceptable additives, e.g. one or more binders, e.g. polyvinylpyrrollidone, and one or more glidants, e.g. colloidal silicon dioxide, e.g. In a high shear mixer;
(ii) adding a solvent, e.g. ethanol, e.g. 94% (w/w), subjecting the mixture to wetting/kneading, e.g. In a high shear mixer, wet-granulating using, e.g., a rotating impeller, and drying, e.g. in a fluidized bed dryer;
(iii) adding pharmaceutically acceptable additives, e.g. sieved additives, e.g. one or more fillers, e.g. lactose, one or more disintegrants, e.g. crosslinked polyvinylpyrrolidone, one or more lubricants, e.g. magnesium stearate, and mixing, e.g. in a container mixer; and
(iv) subjecting the mixture obtained in step (iii) to compression, e.g. in a conventional tabletting machine, e.g. in an EK-0 Korsch eccentric tabletting machine or a rotary tabletting machine, preferably a rotary machine, e.g. at a compression greater than 5 kN.

A tablet according to the present invention containing e.g. about 190 mg of mycophenolate salt, and appropriate additives in appropriate quantities is preferably made by a process wherein the compression force used to produce the tablet is of from about 15 to about 25 kN, preferably about 20 kN. Appropriate additives in appropriate quantities for this active agent may be 45 mg lactose, 6.6 mg anhydrous colloidal silica, 3.25 mg magnesium stearate, 20 mg PVP, 10.25 mg maize starch, and 32.5 mg crosslinked PVP. For a tablet containing e.g. about 385 mg of mycophenolate salt the compression force used to produce the tablet is of from about 15 to about 35 kN, preferably about 20 kN or 30 kN. The particular minimum compression force is dependent on the active agent content in any given formulation and therefore also depends on the amount and nature of the additives present.

The minimum compression force may be determined for other formulations using routine experimentation

The tablet cores may vary in shape and be, for example, round, oval, oblong, cylindrical or any other suitable shape. A characteristic of tablets according to the invention is their small size having regard to the amount of MPA or mycophenolate salt contained therein.

In a preferred embodiment of the invention tablets obtained by the compression method described above are round or oval. The edges of the tablets may be bevelled or rounded.

In a particularly preferred embodiment of the invention a tablet is compressed into a round shape of dimensions diameter height 10.0-10.2 mm: 3.9 mm; or of a tablet having an oval shape of dimensions length:width:height 17.0-17.2:6.7-6.9:5.9 mm.

The tablets of the invention comprising e.g. about 190 mg of mycophenolate salt, may furthermore have a hardness of from about 40 to about 140 N, e.g. about 60 to about 110 N, preferably about 90 N. The tablets of the invention comprising e.g. about 385 mg of mycophenolate salt, may furthermore have a hardness of from about 90 to about 230 N, e.g. about 110 to about 210 N, preferably about 160 N. Tablet hardness is preferably determined according to the standard test e.g. for example using a Schleuniger 6D tablet testing device.

If desired, tablets prepared according to a process as specified above may be coated in e.g. a film-coating machine, e.g. a perforated pan coater, e.g. according to a coating Process as specified above, e.g. with a coating material, comprising e.g. hydroxypropyl methylcellulose phthalates, and pigments dissolved/dispersed in a solvent or a mixture of solvents, e.g. a non-aqueous solvent, e.g. ethanol 94% (w/w)/acetone mixture, to obtain, e.g., a film-coated tablet.

In a particularly preferred embodiment of the invention a coated tablet may have a round shape of dimensions diameter:height of about 10.1-10.7 mm:about 4.2 mm; or an oval shape of dimensions length:width:height of about 17.2-18.0:about 6.9-7.5:about 6.3 mm.

The tablets of the invention may furthermore be coloured, any coating colourless or coloured, and the tablets or coating marked so as to impart an individual appearance and to make them instantly recognizable. The use of dyes can serve to enhance the appearance as well as to identify the compositions. Dyes suitable for use in pharmacy typically include carotinoids, iron oxides, titanium dioxides, indigotine, e.g. indigotine lake, or chlorophyll. Preferably the tablets of the invention are marked using an imprint code.

Procedures which may be used may be conventional or known in the art or based on such procedures e.g those described in L. Lachman et al. The Theory and Practice of Industrial Pharmacy, 3rd Ed, 1986, H. Sucker et al. Pharmazeutische Technologie, Thieme, 1991, Hagers Handbuch der pharmazeutischen Praxis, 4th Ed. (Springer Verlag, 1971) and Remington's Pharmaceutical Sciences, 13th Ed., (Mack Publ., Co., 1970) or later editions.

The compositions of the invention are useful as immunosuppressants as indicated by standard tests.

The activity and characteristics of the compositions of the invention may be indicated in standard
a) clinical trials, e.g. observing the first acute rejection episodes or treatment failure six months after transplant of kidneys or maintaining a rejection-free state within 6 months after initiation of treatment with the invention. The compositions of the invention are administered at a dose in the range of 0.5 to 2.0 g/day e.g. about 1.5 g/day and decrease the acute rejection rates when administered during the period around transplant surgery, and maintain a rejection-free state in patients who are 3 months or more after transplantation. Thus the compositions of the invention may be administered during the initial 72 hours after transplantation at dose of about 0.5 g administered twice a day in combination with a conventional steroid and cyclosporin, e.g. as NEORAL® for which the cyclosporin dose is the conventional dose e.g. ca 8±3 mg/kg for renal transplants. The steroid dose is to be administered at about 2.5 mg/kg for 4 days after transplant, 1 mg/kg thereafter for 1 week, 0.6 mg/kg thereafter for 2 weeks thereafter 0.3 mg/kg for 1 month for prednisone, and in
b) animal trials e.g. observing the kidney allograft reaction in rat. In this test one kidney from a female fisher 344 rat is transplanted onto the renal vessel of a unilaterally (left side) nephrectomized WF recipient rat using an end-to-end anastomosis. Ureteric ananstomosis is also end-to-end. Treatment commences on the day of transplantation and is continued for 14 days. A contralateral nephrectomy is done seven days after transplantation, leaving the recipient relying on the performance of the donor kidney. Survival of the graft recipient is taken as the parameter for a functional graft. Typical doses of the compositions of the invention are from about 1 to 30 mg/kg p.o.

The compositions of the invention are particularly useful for the following conditions:
a) Treatment or prevention of organ, tissue or cellular allograft or xenograft transplant rejection, e.g. for the treatment of recipients of e.g. heart, lung, combined heart-lung, liver, kidney, bowel, pancreatic, skin, pancreatic islet cell, neural cell or corneal transplant; including treatment or prevention of acute rejection; treatment and prevention of hyperacute rejection, e.g. as associated with xenograft rejection; and treatment or prevention of chronic rejection, e.g. as associated with graft-vessel disease. The compositions of the invention are also indicated for the treatment or prevention of graft-versus-host disease, such as following bone marrow transplantation.
b) Treatment or prevention of autoimmune diseases, e.g. Immune-mediated diseases and inflammatory conditions, in particular inflammatory conditions with an etiology including an immunological component such as arthritis (for example rheumatoid arthritis, arthritis chronica progrediente and arthritis deformans) and rheumatic diseases. Specific immune-mediated disease for which the compositions of the invention may be employed include, autoimmune hematological disorders, including, but not limited to hemolytic anaemia, aplastic anaemia, pure red cell anaemia and idiopathic thrombocytopenia), systemic lupus erythematosus, polychondritis, scierodoma, Wegener granulosis, dermatomyositis, polymyositis, chronic active hepatitis, primary bilary cirrhosis, myasthenia gravis, psoriasis, Steven-Johnson syndrome, pemphigus, idiophatic sprue, inflammatory bowel disease (including e.g. ulcerative colitis and Crohn's disease), endocrine opthalmophathy, Graves disease, sarcoidosis, multiple sclerosis, juvenile diabetes (diabetes mellitus type I), non-infectious uveitis (anterior and posterior), keratoconjunctivitis sicca and vernal keratoconjunctivitis, interstitial lung fibrosis, psoriatic arthritis, vasculitis, glomerulonephritides (with and without nephrotic syndrome, e.g. including idiophatic nephrotic syndrome or minimal change nephropathy) and juvenile dermatomyositis.

Appropriate dosage of the compositions of the invention will of course vary, e.g. depending on the condition to be treated (for example the disease type or the nature of resistance), the MPA salt used, the effect desired and the mode of administration.

In general however satisfactory results are obtained on administration e.g. orally at dosages on the order of from about 1 to about 30 mg salt per kg animal body weight per day, administered once or in divided doses up to 4 times per day. Suitable daily dosages for patients are thus in the order of 200 mg to 3 g p.o. salt e.g. from about 50 to 100% that of mycophenolate mofetil. For the preferred mono sodium salt the dosage of the salt is about two thirds that of mycophenolate mofetil.

The bioavailability characteristics of the compositions of the invention may be determined in conventional manner, e.g. by oral administration to beagle dogs. Dosages are typically 50 mg salt/animal e.g. ca 3-5 mg salt/kg animal body weight. Dogs are adult (ca. 10 kg, e.g. 6-14 kg) and fasted. Three hours after administration ca. 200 g food is administered. Blood samples are taken from the cephalic vein, before administration and 10, 30, and 45 minutes, 1, 1.5, 2, 3, 4, 6, 8, 12, and 24 hours, after administration. Plasma levels of free MPA are determined by HPLC analysis (with UV detection).

The compositions of the invention comprising a therapeutically effective amount of mycophenolic acid or mycophenolate salt may be administered as the sole active ingredient or with another immunosuppressant e.g. together with simultaneous or separate administration of other immunosuppressants, for example, in immunosuppressive applications such as prevention or treatment of graft vs. host disease, transplant rejection, or immune-mediated diseases. For example, the compositions of the invention may be used in combination with cyclosporins or ascomycins, or their immunosuppressive analogs, e.g. cyclosporin A, FK-506 (tacrolimus), etc., rapamycin or a derivative thereof, e.g. 40-O-(2-hydroxyethyl)-rapamycin, a derivative as disclosed e.g. in WO 95/14023 and 99/15530, e.g. ABT578, or rapalogs as disclosed e.g. in WO 98/02441 and WO01/14387, e.g. AP23573; a lymphocyte homing agent, e.g. FTY720 (2-amino-2-[2-(4-octylphenyl)ethyl]propane-1,3-diol in free form or in a pharmaceutically salt form, e.g. the hydrochloride), corticosteroids; cyclophosphamide; azathioprine; methotrexate; brequinar; leflunomide; mizoribine; deoxyspergualin; or immunosuppressive monoclonal antibodies, e.g., monoclonal antibodies to leukocyte receptors, e.g. MHC, CD2, CD3, CD4, CD7, CD25, CD28, CTLA4, B7, CD40, CD45, or CD58 or to their ligands; or other immunomodulatory compounds. A preferred combination comprises a composition of the invention and rapamycin or a derivative thereof, e.g. as indicated above, e.g. 40-O-(2-hydroxyethyl)-rapamycin, and/or a lymphocyte homing agent, e.g FTY720.

Accordingly in a further aspect the present invention provides a method of immunosuppressing a subject which comprises administering a solid dosage form according to the invention, e.g. a tablet, to a subject in need of such immunosuppression, optionally with the simultaneous, sequential or separate administration of another immunosuppressant or immunomodulatory compound, e.g. as disclosed above.

When the compositions of the invention are co-administered with such other immunosuppressants the dosages of the other immunosuppressants may be reduced e.g. to one-half to one-third their dosages when used alone.

Representative doses for ciclosporin to be used are e.g. 1 to 10, e.g. 1 to 2 mg/kg/day.

The following Examples serve to illustrate the invention.

| | Example 1 | | |
|---|---|---|---|
| | mg (% of core; | | Example 2 |
| | % coated tablet) | | mg |
| Tablet content | | | |
| Mycophenolate sodium | 192.4¹ | (62.1; 54.2) | 384.8² |
| Lactose, anhydr. (1.1) | .45 | (14.5; 12.7) | 90 |
| Crospovidone ® (1.2) | 32.5 | (10.5; 9.2) | 65 |
| Povidone ® K30 (1.3) | 20 | (6.5; 5.6) | 40 |
| Maize starch (1.2) | 10.25 | (3.3; 2.9) | 20.5 |
| Colloidal silicon dioxide, | 6.6 | (2.1; 1.9) | 13.2 |
| anhydr. (1.4) | | | |
| Magnesium stearate (1.5) | 3.25 | (1; 0.9) | 6.5 |
| Total (core) | 310 | (100; 87.4) | 620 |
| Enteric coating | | | |
| HPMCP HP50 | 42 | (—; 11.8) | 65 |
| Iron oxide yellow | 0.078 | | 0.167 |
| Iron oxide red | — | | 0.167 |
| Titanium dioxide | 2.883 | (—; 0.8) | 4.666 |
| Indigotine lake | 0.039 | | — |
| Total (coating) | 45 | (—; 12.6) | 70 |
| Total coated tablet | 355 | (145/100) | 690 |
| Ethanol 94% (w/w), den.³ | | | |
| (Granulation and coating liquid) | | | |
| Acetone³ (Coating liquid) | | | |

¹Equivalent to 180 mg mycophenolic acid
²Equivalent to 360 mg mycophenolic acid
³Lost during the film coat drying stage

### Method

The tablet ingredients mycophenolate sodium, Povidone® K30, silica, colloidal anhydrous are

(i) mixed;
(II) wet-granulated using ethanol 94% (w/w);
(iii) mixed with lactose anhydrous, maize starch, Crospovidone®, and magnesium stearate; and compressed to tablets preferably about 20 kN.
(iv) The tablets are coated in a perforated pan coater with a solution of the coating ingredients in ethanol (with 5% isopropanol)/acetone. The tablets meet the enteric coating test described herein and do not disintegrate within 2 hours in artificial gastric Juices (pH 1, HCl). The compositions are stable, e.g. for 2 years with a disintegration of less than 5% mycophenolic add content at room temperature.

By following above procedure, following tablets (coated and uncoated) may be obtained:

| | |
|---|---|
| Tablet content | mg |

Example 3

| | |
|---|---|
| Mycophenolate sodium | 342* |
| Lactose, anhydr. (1.1) | 80 |
| Crospovidone ® (1.2) | 57.80 |
| Povidone ® K30 (1.3) | 35.50 |
| Maize starch (1.2) | 18.20 |
| Colloidal silicon dioxide, | 11.70 |
| anhydr. (1.4) | |
| Magnesium stearate (1.5) | 5.80 |
| Total (core) | 551 |
| Enteric coating | |
| HPMCP HP50 | 60 |
| Total tablet | 611 |

*equivalent to 320 mg MPA

Example 4

A granulate is manufactured using the organic wet granulation process,
ethanol abs. being used as granulation liquid.

| | |
|---|---|
| Mycophenolate sodium | 192.3* |
| Lactose 200 mesh (1.1) | 147.7 |
| Crosscarmellose (1.2) | 100.0 |
| Povidone ® K30 (1.3) | 35.0 |
| Colloidal silicon dioxide, | 20.0 |
| anhydr. (1.4) | |
| Magnesium stearate | 5.0 |
| Total | 500 |

This granulate may be further mixed with additives and compressed
into tablets.

| | |
|---|---|
| Enteric coating | |
| HPMCP HP50 | 60 |
| Total Tablet | 560 |

*equivalent to 190 mg MPA

1-10. (canceled)

11. A tablet comprising an enteric coating and a pharmacologically effective amount of mycophenolate sodium salt, wherein the mycophenolate sodium salt is present in an amount of from at least about 45% to about 80% by weight based on the total weight of the tablet including the enteric coating.

12. The tablet of claim 11, additionally comprising:
one or more pharmaceutically acceptable additives suitable for the preparation of tablets by compression methods.

13. A tablet according to claim 11 containing mycophenolate sodium salt in crystalline form.

14. A tablet according to claim 11 containing mycophenolate crystalline mono sodium salt in anhydrous form.

15. A tablet according to claim 11 wherein the mycophenolate sodium salt is present in an amount of from at least about 50% to about 65% by weight based on the total weight of the solid dosage form including the enteric coating.

16. A process for the preparation of a tablet according to claim 12, which process comprises
(i) mixing the mycophenolate sodium salt and pharmaceutically acceptable additives,
(ii) subjecting a mixture obtained in step (i) to granulation,
(iii) compressing the granules obtained in step (ii) and pharmaceutically acceptable additives to form the tablet.
step (ii) being optional.

17. A granule produced by a process as set out in steps (i) and (ii) according to claim 16.

18. A tablet produced by a process according to claim 16.

19. A method of immunosuppressing a subject which comprises administering a tablet according to claim 11 to a subject in need of such immunosuppression, optionally with the simultaneous, sequential or separate administration of another immunosupressant.
